(19) United States
(12) Patent Application Publication
(10) Pub. No.: US 2010/0247470 A1
(43) Pub. Date: Sep. 30, 2010

(54) Mascara Compositions And Methods For Treating Lashes

(76) Inventors: Francis Martin Friel, Merrick, NY (US); Ariene G. Ting-Jenulis, East Northport, NY (US); Tatyana R. Tabakman, Brooklyn, NY (US); John R. Castro, Huntington Station, NY (US)

Correspondence Address: THE ESTEE LAUDER COS, INC, 155 PINELAWN ROAD, STE 345 S, MELVILLE, NY, 11747, US

(21) Appl. No.: 12/414,816

(22) Filed: Mar. 31, 2009

(51) Int. Cl. A61K 8/92; A61Q 1/10

(52) U.S. Cl. 424/70.7

(57) ABSTRACT

A water and oil emulsion mascara composition comprising a plurality of water soluble or dispersible film forming polymers in the aqueous phase and being structured by one or more emulsifiers in solid or semi-solid form.

### TECHNICAL FIELD

The invention is in the field of color cosmetic compositions for application to keratinous surfaces, including eyelashes and hair.

### BACKGROUND OF THE INVENTION

Mascaras are used by women to lengthen, curl and accentuate the lashes. Lash accentuation dates back to the time of Cleopatra. Mascara formulas have dramatically improved since then, and cosmetics companies are always looking for improved formulas that will provide the typical benefits desired by consumers. Included among those are lash darkening, lengthening, thickening, and curling.

In addition, one common problem with mascaras relates to viscosity increase with time. The waxes traditionally used to thicken mascara formulas work well for this purpose, however, when mascara is used the constant opening and closing of the container causes liquid evaporation and thickening of the product with time. This in turn may cause the mascara to be clumpy when applied. In addition, because waxes are typically opaque solids, mascaras containing such waxes exhibit more muted colors when compared with similar formulas containing no waxes or clear polymeric waxes such as polyamides or silicone polyamides. While cosmetic formulators can arrive at mascara formulas with intense color by using clear waxes, there are not that many of them available. In addition, clear polymer waxes such as polyamides or silicone polyamides exhibit other formulation difficulties, particularly with the oil in water emulsion form common to most mascaras.

It has been discovered that mascaras formulated with a plurality of water soluble or dispersible film forming polymers, emulsifiers that are solid or semi-solid at room temperature (e.g. 25° C.), particularly when in the oil in water emulsion form, provide excellent mascara formulas that lengthen, color, and curl lashes and provide a more intense color than standard wax based formulas. In addition, these mascara formulas are very suitable for use with heated mascara applicators in that they may be repeatedly heated and cooled without significant viscosity change over time.

It is an object of the invention to provide emulsion mascara formulas that lengthen, color, and curl lashes, provide improved pigment intensity, and are thermodynamically stable.

It is a further object of the invention to provide mascara formulas in the water and oil emulsion form that contain a plurality of water soluble or dispersible film forming polymers, emulsifiers in the solid or semi-solid form, which may be substantially free of opaque animal, vegetable or mineral waxes.

It is a further object of the invention to provide a mascara formula in the oil in water emulsion form comprising a plurality of water soluble or dispersible film forming polymers, at least one solid or semi-solid emulsifier, and fibers.

It is a further object of the invention to provide a mascara formula in the oil and water emulsion form comprising a plurality of water soluble or dispersible film forming polymers, at least one solid or semi-solid emulsifier, which composition is substantially free of oil phase film forming polymers.

It is a further object of the invention to provide a method for lengthening, coloring, or curling eyelashes by applying the mascara composition of the invention.

### SUMMARY OF THE INVENTION

The invention is directed to a substantially opaque-wax-free water and oil emulsion mascara composition comprising a plurality of water soluble or dispersible film forming polymers in the aqueous phase and being structured by one or more emulsifiers in solid or semi-solid form.

The invention is further directed to a water and oil emulsion mascara composition comprising an aqueous phase containing a plurality of water soluble or dispersible film forming polymers and being substantially free of oil phase film forming polymers, wherein the composition is structured by one or more emulsifiers in solid or semi-solid form.

The invention is further directed to a water and oil emulsion mascara composition comprising an aqueous phase containing a plurality of water soluble or dispersible film forming polymers, fibers, and being structured by at least one emulsifier in the solid or semi-solid form.

The invention is further directed to a method for lengthening, coloring, or curling lashes by applying a substantially opaque-wax-free mascara composition comprised of a water and oil emulsion containing an aqueous phase with a plurality of water soluble or dispersible film forming polymers and being structured by at least one emulsifier in the solid or semi-solid form.

### DETAILED DESCRIPTION

The composition is in the form of an emulsion which may be a water in oil or oil in water emulsion. The amount of water present may range from about 0.1 to 99%, preferably from about 0.5 to 80%, more preferably from about 1 to 75% with all percentages mentioned herein being percentages by weight unless otherwise indicated. The oil present may range from about 0.1 to 95%, preferably from about 0.5 to 90%, more preferably from about 1 to 80% of the composition. In one preferred embodiment the mascara composition is an oil in water emulsion.

The term “opaque wax” means that the wax is not transparent or translucent; e.g. that it does not permit passage of light.

The term “plurality” means more than one.

The term “structured” means that the viscosity of the composition is increased.

The term “substantially free of” means that the composition contains less than about 5%, preferably less than about 3%, more preferably less than about 1%, most preferably none, of the ingredient mentioned with respect to the phrase, with all percentages mentioned herein percentages by weight unless otherwise indicated.

### I. The Water Soluble or Dispersible Film Forming Polymers

The composition comprises an aqueous phase containing a plurality of water soluble or dispersible film forming polymers. Suggested ranges are from about 0.1 to 95%, preferably from about 0.5 to 50%, more preferably from about 0.5 to 20% of the composition. Suitable film forming polymers may include synthetic or natural polymers. Examples of synthetic polymers include those obtained from ethylenically unsaturated monomers such as acrylic acid, methacrylic acid or their simple C₁₋₁₀ alkyl esters; or homo- or copolymers of acrylamide, ammonium acrylate, acrylates, methacrylates, butyl acrylamide, ethyl hexylacrylate, octylacrylamide, vinyl neodecanoate, stearyl acrylate, methyl methacrylates, adipic acid, butadiene, vinyl acetate, vinyl alcohol, vinyl pyrrolidone, acrylic acid, isobutyl acrylate, isobutyl methacrylate, sodium acrylate, hydroxyethyl methacrylate, styrene, ammonium acrylate, butylacrylamide, sodium acrylate, stearyl acrylate, maleic anhydride, ethylene, propylene, isophorone diisocyanate, isocyanate, diisocyanate, phthalic acid, ethylmethacrylate, tosylamide, and so on.

Such film formers may include Acrylamide/ammonium acrylates copolymer, acrylamides copolymer, acrylamide/sodium acrylate copolymer, acrylamide/sodium aeryloyldimethyltaurate/acrylie acid copolymer, acrylates/acrylamide copolymer, acrylates/ammonium methacrylate copolymer, acrylates/behenyl methacrylate/dimethicone methacrylate copolymer, acrylates/bis-hydroxypropyl dimethicone crosspolymer, acrylates/t-butylacrylamide copolymer, acrylates/C12-22 alkyl methacrylate copolymer, acrylates acrylates/dimethylaminoethyl methacrylate copolymer, acrylates/ethylhexylacrylate copolymer, acrylates/ethylhexyl acrylate/styrene copolymer, acrylates/hydroxyethyl acrylate/methoxyethyl acrylate copolymer, acrylates/stearyl acrylate/methacrylate copolymer, acrylates/VP copolymer, acrylates/VA copolymer, acrylates/VA crosspolymer, acrylates/VP/dimethylaminoethyl methacrylate copolymer, adipic acid, diethylene glycol/glycerin crosspolymer, allyl stearate/VA copolymer, ammonium acrylates copolymer, ammonium acrylates/ethylhexyl acrylate copolymer, ammonium styrene/acrylates copolymer, ammonium VA/acrylates copolymer, ammonium VA/crotonic acid copolymer, behenyl methacrylate/perfluorooctylethyl methacrylate copolymer, butyl acrylate/ethylhexyl methacrylate copolymer, butyl acrylate/glycol dimethacrylate copolymer, butyl acrylate/styrene acrylate copolymer, isobutylene/MA copolymer, lauryl acrylate/VA copolymer, Polyacrylate 1, 2, 5, 6, 8, 9, 12, 13, 14, 15, 16, 17, 18, 19; polyacrylic acid, Polyamide 1, Polyamide 2, Polyester 1, 2, 3, 4, 5; polyethylacrylate, polymethacrylamide, Polyurethane 1, 2, 4, 5, 6, 7, 8, 10, 11, 12, 13, 14, 15, 16, 17, 20, or 21; polyvinyl acetate, polyvinyl alcohol, PVP, PVM/MA copolymer, PVM/MA decadiene copolymer, PVP/MA copolymer, PVP/VA/vinyl propionate copolymer, PVP/VA/itaconate copolymer, PVM/MA copolymer, sodium acrylates copolymer, sodium styrene/acrylates copolymer, styrene/MA copolymer, styrene/VA copolymer, VA/crotonates copolymer, VP/dimethylaminoethylacrylate copolymer, and the like.

More specific examples of suitable film forming polymers include, but are not limited to methyl methacrylate crosspolymer; Polyimide-1; Disteareth-100 IPDI copolymer; Hydrolyzed Wheat Protein/Cystine Bis-PG-Propyl Silanetriol Copolymer; PVA; Sodium Polystyrene Sulfonate; Hydrolyzed Wheat Protein/PVP Crosspolymer; Acrylates copolymer; and so on. Further examples are set forth in the C.T.F.A. Cosmetic Ingredient Dictionary and Handbook, Eleventh Edition, 2006, Volume 3, pages 2779-2783, which are hereby incorporated by reference in their entirety.

### II. Oils

The compositions of the invention are preferably in emulsion form, and may be in the form of a water in oil or oil in water emulsion. Suitable oils may be volatile or nonvolatile; or organic, silicone, or hydrocarbon oils. The term “oil” when used herein generally means an oil that is pourable at room temperature (25° C.). The oils may be present ranging from about 0.1 to 99%, preferably from about 0.5 to 90%, more preferably from about 1 to 85%.

Suitable volatile silicones include branched silicones such as methyl trimethicone (1.5 centistokes); linear volatile silicones such as hexamethyldisiloxane (0.5 cs), octamethyltrisiloxane (1.0 cs), decamethyltetrasiloxane (1.5 cs), dodecamethylpentasiloxane (2.0 cs); cyclic volatile silicones generically referred to as “cyclomethicone” which may be cyclotetrasiloxane, cyclopentasiloane, or cyclohexasiloxane. Suitable volatile hydrocarbons include C9-12 paraffinic hydrocarbons such as isododecane, isohexadecane and the like.

Also suitable are nonvolatile silicones such as dimethicone, phenyl trimethicone, phenyldimethicone, diphenyldimethicone, trimethylsiloxyphenyldimethicone, cetyl dimethicone, and the like.

Examples of nonvolatile organic oils that may be used include naturally occurring plant oils such as soybean, castor seed, wheat germ, grape seed, almond, lavender, and so on.

Examples of nonvolatile organic oils may also include synthetic or naturally occurring mono-, di-, or triesters including esters of C₆₋₃₀ fatty carboxylic acids and mono-, di-, or polyhydric alcohols having from about 2 to 40 carbon atoms. Examples include isostearyl malate, diisostearyl malate, isononyl isononanoate, myristyl myristate, diglyceryl diisostearate, glyceryl stearate, glyceryl isostearate, polyglyceryl-3 isostearate, polyglyceryl-3 diisostearate, and so on.

Examples of nonvolatile hydrocarbons include squalane, squalene, eicosane, and the like.

### III. Solid or Semi-Solid Emulsifiers

The solid or semi-solid emulsifiers used in the compositions of the invention may be present in amounts ranging from about 0.01 to 50%, preferably from about 0.05 to 45%, more preferably from about 0.1 to 35%. The emulsifiers are solid or semi-solid at room temperature (e.g. 25° C.) and are used to increase the viscosity, or thicken, the composition. Suitable solid or semi-solid emulsifiers may be synthetic or naturally occurring and include alkoxylated alcohols including alkoxylated fatty C₁₂₋₄₀ straight or branched chain alcohols, alkoxylated naturally occurring hydrogenated or non-hydrogenated solid or semi-solid oils, alkylene glycols, and so on. More specific examples include alkoxylated fatty alcohols such as laureth, steareth, isosteareth, beheneth, etc., having a number of ethylene oxide repeat units ranging from 2 to 500; polyalkylene glycols, including polyethylene glycols, having repeating alkylene oxide or ethylene oxide units ranging from about 5 to 200; ethoxylated hydrogenated emulsifiers derived from natural sources, e.g. castor oil; ethoxylated branched chain fatty ethoxylated emulsifiers; and the like. More specific examples of suitable solid or semi-solid emulsifiers include PEG-20, Steareth-2, Steareth-21, Steareth-100, PEG-40 hydrogenated castor oil, PEG-6 decyltetradeceth-30, and the like.

### IV. Opaque Waxes

In one embodiment the composition is substantially free of opaque waxes. The opaque waxes that the composition may be substantially free of include beeswax, carnauba, ozokerite, synthetic wax, bayberry wax, candelilla, acacia, ceresin, cetyl esters, flower wax, citrus wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and so on.

### V. Humectants

It may also be desirable to include one or more humectants in the composition. If present, such humectants may range from about 0.001 to 25%, preferably from about 0.005 to 20%, more preferably from about 0.1 to 15% by weight of the total composition. Examples of suitable humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄alkylene glycols, most particularly butylene glycol.

### VI. Pigments and Particulate Powders

The composition also preferably contains one or more pigments. The pigments may be organic or inorganic. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable. Suggested ranges may be from about 0.001 to 40%, preferably from about 0.005 to 35%, more preferably from about 0.01 to 30%.

Suitable powders include colored or non-colored (for example white) non-pigmented powders. They may be present in amounts ranging from about 0.01 to 40%, preferably from about 0.05 to 35%, more preferably from about 0.1 to 30% of the composition. Suitable non-pigmented powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

### VII. Fibers

The composition may also contain fibers, which will provide length and accentuate lashes. If present such fibers may range from about 0.01 to 20%, preferably from about 0.05 to 15%, more preferably from about 0.1 to 10% of the composition. Fibers may be natural or synthetic and include nylon, silk, polyethylene, polyethylene terephthalate, polypropylene, microdenier fibers, and so on.

### VIII. Oil Phase Film Formers

In one embodiment the composition is substantially free of oil phase film forming polymers. Examples of oil phase film forming polymers that the composition may be free of include silicone resins such as trimethylsiloxy silicate, an MT resin such as methylsilsesquioxane, polyamides, and so on.

The compositions will be further described in connection with the following examples which are set forth for the purposes of illustration only.

### EXAMPLE 1

Oil in water emulsion mascara compositions were prepared as follows:

| Ingredients | w/w % | | |
|---|---|---|---|
| Water | QS100 | QS100 | QS100 |
| Butylene glycol | 5.00 | 3.00 | 3.00 |
| Ethylhexyl glycerin | | | 0.30 |
| Water/acrylates copolymer | | | 10.00 |
| Polyglyceryl-3 diisostearate liquid | 0.50 | | |
| Iron oxides | 12.00 | 6.00 | 6.00 |
| Kaolin powder | 4.00 | 3.00 | 6.00 |
| Polyisobutene | | | 6.00 |
| Acrylates/octylacrylamide copolymer | | | 5.00 |
| Methyl methacrylate crosspolymer dispersion | 6.00 | 9.00 | |
| Mica | 2.00 | | |
| Mica/Methyl methacrylate crosspolymer | | | 3.00 |
| Silica | | | 1.00 |
| Polyquaternium-10 | 0.70 | | |
| Steareth-100/Disteareth-100 IPDI | 1.00 | 3.00 | 2.25 |
| copolymer viscous liquid | | | |
| Polyimide-1 | | 12.00 | |
| Sodium polystyrene sulfonate (FF polymer | | 6.00 | |
| Simethicone (liquid) | 0.10 | 0.10 | 0.10 |
| Biosaccharide gum (skin conditioning agent) | 0.10 | | |
| Sodium dehydroacetate | 0.10 | 0.10 | 0.10 |
| Bentonite (thickening agent) | | 1.25 | 1.25 |
| Disodium EDTA (preservative) | 0.10 | 0.10 | 0.10 |
| Hydrogenated castor oil (solid) | 5.00 | | |
| Steareth-2 (solid) | 2.25 | | |
| Steareth-21 (solid) | 0.75 | 3.50 | 3.50 |
| PEG-6 decyltetradeceth-30 | 0.50 | | |
| PEG-40 hydrogenated castor oil (solid) | 0.50 | | |
| Ricinus Communic (Castor) seed oil (Liquid) | 2.00 | | |
| PEG-20 (Solid) | 2.00 | 3.00 | 3.00 |
| PVP | | | 0.50 |
| PTFE | 2.00 | | |
| Polyvinyl alcohol (Film forming polymer) | | 3.00 | 3.00 |
| Nylon 6/silica | 0.50 | | |
| Water/hydrolyzed wheat protein/ | 1.00 | | |
| PVP crosspolymer | | | |
| Water/hydrolyzed wheat protein | | 1.00 | |
| Water/hydrolyzed wheat protein/ | | 7.00 | |
| cystine bis-PG-propyl silanetriol | | | |
| copolymer (skin conditioning agetnt) | | | |
| Water/polyaminopropyl biguanide | 0.20 | 0.10 | 0.05 |
| Water/acrylates copolymer, butylene | 5.00 | | |
| glycol/sodium laureth sulfate | | | |
| Phenoxyethanol/caprylyl glycol/potassium | | 0.75 | |
| sorbate/water/hexylene glycol | | | |
| Phenoxyethanol | | 0.40 | 0.40 |
| Phenoxyethanol/caprylyl glycol/potassium | | | 0.85 |
| sorbate/water/hexylene glycol | | | |
| Aminomethyl propanediol | | 0.05 | |

The composition was prepared by combining the water phase and oil phase ingredients separately, then mixing the phases to emulsify and form a mascara composition.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A substantially opaque-wax-free water and oil emulsion mascara composition comprising a plurality of water soluble or dispersible film forming polymers in the aqueous phase and being structured by one or more emulsifiers in solid or semi-solid form.

2. The composition of claim 1 wherein the emulsifiers are alkoxylated alcohols, alkoxylated fatty C₁₂₋₂₀ straight or branched chain alcohols, or mixtures thereof.

3. The composition of claim 1 wherein the emulsifier is selected from PEG-20, Steareth-2, Steareth-21, PEG-40 hydrogenated castor oil, or mixtures thereof.

4. The composition of claim 1 further comprising fibers.

5. The composition of claim 1 wherein the water soluble or dispersible film forming polymers are homo- or copolymers of acrylamide, ammonium acrylate, acrylates, methacrylates, butyl acrylamide, ethyl hexylacrylate, octylacrylamide, vinyl neodecanoate, stearyl acrylate, methyl methacrylates, adipic acid, butadiene, vinyl acetate, vinyl alcohol, vinyl pyrrolidone, acrylic acid, isobutyl acrylate, isobutyl methacrylate, sodium acrylate, hydroxyethyl methacrylate, styrene, ammonium acrylate, butylacrylamide, sodium acrylate, stearyl acrylate, maleic anhydride, ethylene, propylene, isophorone diisocyanate, isocyanate, diisocyanate, phthalic acid, ethylmethacrylate, tosylamide, or mixtures thereof.

6. The composition of claim 5 wherein the film forming polymers are selected from methyl methacrylate crosspolymer; Polyimide-1; Disteareth-100 IPDI copolymer; Hydrolyzed Wheat Protein/Cystine Bis-PC-Propyl Silanetriol Copolymer; PVA; Sodium Polystyrene Sulfonate; Hydrolyzed Wheat Protein/PVP Crosspolymer; Acrylates copolymer: Acrylates/Octyl Acrylamide copolymer; methyl methacrylate crosspolymer; PVP, PVA, or mixtures thereof.

7. The composition of claim 6 further comprising at least one humectant.

8. The composition of claim 6 further comprising at least one non-volatile oil.

9. The composition of claim 8 wherein the non-volatile oil comprises a hydrocarbon.

10. The composition of claim 9 wherein the non-volatile hydrocarbon oil comprises polybutene, polyisobutene, polydecene, or mixtures thereof.

11. The composition of claim 1 wherein the film forming polymers are selected from acrylates copolymer, acrylates/octylacrylamide copolymer, polyvinyl alcohol, disteareth-100 IPDI copolymer, PVA, PVP, methyl methacrylate crosspolymer, sodium polystyrene sulfonate, hydrolyzed wheat protein/PVP crosspolymer, Polyimide-1, and mixtures thereof.

12. A water and oil emulsion mascara composition comprising an aqueous phase containing a plurality of water soluble or dispersible film forming polymers and being substantially free of oil phase film forming polymers, wherein the composition is structured by one or more emulsifiers in solid or semi-solid form.

13. The composition of claim 12 wherein the plurality of water soluble or dispersible film forming polymers are homo- or copolymers of acrylamide, ammonium acrylate, acrylates, methacrylates, butyl acrylamide, ethyl hexylacrylate, octylacrylamide, vinyl neodecanoate, stearyl acrylate, methyl methacrylates, adipic acid, butadiene, vinyl acetate, vinyl alcohol, vinyl pyrrolidone, acrylic acid, isobutyl acrylate, isobutyl methacrylate, sodium acrylate, hydroxyethyl methacrylate, styrene, ammonium acrylate, butylacrylamide, sodium acrylate, stearyl acrylate, maleic anhydride, ethylene, propylene, isophorone diisocyanate, isocyanate, diisocyanate, phthalic acid, ethylmethacrylate, tosylamide, or mixtures thereof.

14. The composition of claim 12 wherein the plurality of water soluble or dispersible film forming polymers are selected from acrylamide/ammonium acrylates copolymer, acrylamides copolymer; acrylamide/sodium acrylate copolymer; acrylamide/sodium acryloyldimethyltaurate/acrylic acid copolymer; acrylates/acrylamide copolymer; acrylates/ammonium methacrylate copolymer; acrylates/behenyl methacrylate/dimethicone methacrylate copolymer; acrylates/bis-hydroxypropyl dimethicone crosspolymer; acrylates/t-butylacrylamide copolymer; acrylates/C12-22 alkyl methacrylate copolymer; acrylates acrylates/dimethylaminoethyl methacrylate copolymer; acrylates/ethylhexylacrylate copolymer; acrylates/ethylhexyl acrylate/styrene copolymer; acrylates/hydroxyethyl acrylate/methoxyethyl acrylate copolymer; acrylates/stearyl acrylate/methacrylate copolymer; acrylates/VP copolymer; acrylates/VA copolymer; acrylates/VA crosspolymer; acrylates/VP/dimethylaminoethyl methacrylate copolymer; adipic acid diethylene glycol/glycerin crosspolymer; allyl stearate/VA copolymer; ammonium acrylates copolymer; ammonium acrylates/ethylhexyl acrylate copolymer; ammonium styrene/acrylates copolymer; ammonium VA/acrylates copolymer; ammonium VA/crotonic acid copolymer; behenyl methacrylate/perfluorooctylethyl methacrylate copolymer; butyl acrylate/ethylhexyl methacrylate copolymer; butyl acrylate/glycol dimethacrylate copolymer; butyl acrylate/styrene acrylate copolymer; isobutylene/MA copolymer; lauryl acrylate/VA copolymer; Polyacrylate 1; Polyacrylate 2; Polyacrylate 5; Polyacrylate 6; Polyacrylate 8; Polyacrylate 9; Polyacrylate 12; Polyacrylate 13; Polyacrylate 14; Polyacrylate 15; Polyacrylate 16; Polyacrylate 17; Polyacrylate 18; Polyacrylate 19; polyacrylic acid; Polyamide 1; Polyamide 2; Polyester 1; Polyester 2; Polyester 3; Polyester 4; Polyester 5; polyethylacrylate; polymethacrylamide; Polyurethane 1; Polyurethane 2; Polyurethan 4; Polyurethane 5; Polyurethane 6; Polyurethane 7; Polyurethane 8; Polyurethane 10; Polyurethane 11; Polyurethane 12; Polyurethane 13; Polyurethane 14; Polyurethane 15; Polyurethane 16; Polyurethane 17; Polyurethane 20, Polyurethane 21; polyvinyl acetate; polyvinyl alcohol; PVP, PVM/MA copolymer; PVM/MA decadiene copolymer; PVP/MA copolymer; PVP/VA/vinyl propionate copolymer; PVP/VA/itaconate copolymer; PVM/MA copolymer; sodium acrylates copolymer; sodium styrene/acrylates copolymer; styrene/MA copolymer; styrene/VA copolymer; VA/crotonates copolymer; VP/dimethylaminoethylacrylate copolymer; methyl methacrylate crosspolymer; Disteareth-100 IPDI copolymer; Hydrolyzed Wheat Protein/Cystine Bis-PG-Propyl Silanetriol Copolymer; PVA; Sodium Polystyrene Sulfonate; Hydrolyzed Wheat Protein/PVP Crosspolymer: Acrylates copolymer; or mixtures thereof.

14. The composition of claim 13 wherein the solid or semi-solid emulsifier comprises an alkoxylated alcohol.

15. The composition of claim 14 wherein the solid or semi-solid emulsifier comprises an alkoxylated fatty alcohol.

16. The composition of claim 14 further comprising fibers.

17. A method for lengthening, coloring, or curling lashes by applying a substantially opaque-wax-free mascara composition comprised of a water and oil emulsion containing an aqueous phase with a plurality of water soluble or dispersible film forming polymers and being structured by at least one emulsifier in the solid or semi-solid form.

18. The method of claim 17 wherein the composition additionally comprises at least one humectant.

19. The method of claim 17 wherein the composition additionally comprises at least one hydrocarbon oil.

20. The method of claim 19 wherein the hydrocarbon oil comprises polybutene, polyisobutene, polydecene, or mixtures thereof.
